(19) United States
(12) Patent Application Publication
(10) Pub. No.: US 2004/0052755 A1
(43) Pub. Date: Mar. 18, 2004

(54) Cosmetic composition

(75) Inventor: Noriyuki Nakanishi, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO., INC, Tokyo (JP)

(21) Appl. No.: 10/615,780

(22) Filed: Jul. 10, 2003

(30) Foreign Application Priority Data
Jul. 11, 2002 (JP) 2002-202145

(51) Int. Cl. A61K 7/075; A61K 7/08

(52) U.S. Cl. 424/70.31

(57) ABSTRACT

A cosmetic composition which comprises the following ingredients: (A) one or more types of surfactant selected from the group consisting of surfactants which have an oxyethylene group in the molecule (for example, a surfactant represented by [R¹(OCH₂CH₂)ₙ—OSO₃]⁻M⁺ (R¹ represents an C₇₋₂₁ alkyl group and the like, n represents an integer of 1 to 30, and M represents Na, K, NH₄, or triethanolamine)); (B) tert-butanol. In the cosmetic composition, the smell change or the odor generation with passage of time caused by addition of a surfactant having an oxyethylene group in the molecule is suppressed, and thus the cosmetic composition with high storage stability is provided.

### TECHNICAL FIELD

The present invention relates to a cosmetic composition. More specifically, the present invention relates to a cosmetic composition which comprises a surfactant having an oxyethylene group in the molecule and tert-butanol.

### BACKGROUND ART

Surfactants having an oxyethylene group (—CH₂CH₂O—) in the molecule are widely used in cosmetics such as skin washing agents, hair washing agents, and emulsifying agents. For example, alkylether sulfates are used for skin washing agents or hair washing agents, and polyoxyethyleneglyceryl esters and polyoxyethylenealkyl ethers and the like are commonly used as emulsifying agents. However, when a surfactant having an oxyethylene group in the molecule is mixed in cosmetics, a problem arises in that the presence of an oxyethylene group may become a cause of smell change or odor generation with passage of time. Masking with fragrance, formulation with a chelate agent or an antioxidant and the like have so far been employed to solve the problem of the smell change or the odor generation.

### DISCLOSURE OF THE INVENTION

An object of the present invention is to provide a cosmetic composition which comprises a surfactant having an oxyethylene group in the molecule, and wherein the smell change or the odor generation with passage of time are reduced or eliminated. The inventor of the present invention conducted intensive researches to achieve the foregoing object, and as a result, they found that the smell change or the odor generation with passage of time are significantly suppressed by addition of tert-butanol to a cosmetic comprising a surfactant having an oxyethylene group in the molecule. The present invention was achieved on the basis of these findings.

The present invention thus provides a cosmetic composition which comprises the following ingredients:

(A) one or more types of surfactants selected from the group consisting of surfactants which have an oxyethylene group in the molecule;

(B) tert-butanol.

According to preferred embodiments of the aforementioned invention, there are provided the aforementioned cosmetic composition wherein the ratio of the surfactant having an oxyethylene group in the molecule is 1 to 20 weight % based on the total weight of the composition, and the aforementioned cosmetic composition wherein the ratio of tert-butanol is 0.01 to 1,000 ppm based on the total weight of the composition.

According to further preferred embodiment of the present invention, there is provided the aforementioned cosmetic composition wherein the surfactant having an oxyethylene group in the molecule is one or more types of surfactants selected from the group consisting of a surfactant represented by the following general formula (1):

[R¹(OCH₂CH₂)ₙ—OSO₃]⁻M+

(wherein R¹ represents a linear or branched alkyl group having 7 to 21 carbon atoms or a linear or branched alkenyl group having 7 to 21 carbon atoms, n represents an integer of 1 to 30, and M represents Na, K, NH₄, or triethanolamine);

a surfactant represented by the following general formula (2):

R²CO—NH(CH₂CH₂O)ₘH

(wherein R² represents a linear or branched alkyl group having 7 to 21 carbon atoms or a linear or branched alkenyl group having 7 to 21 carbon atoms, and m represents an integer of 1 to 10);

a surfactant represented by the following general formula (3):

R³CO—N (CH₂CH₂OH)₂

(wherein R³ represents a linear or branched alkyl group having 7 to 21 carbon atoms or a linear or branched alkenyl group having 7 to 21 carbon atoms).

In another aspect, the present invention provides a method for suppressing the smell change or the odor generation with passage of time in cosmetics comprising a surfactant having an oxyethylene group in the molecule, which comprises the step of adding tert-butanol to said cosmetics. According to preferred embodiments of the present invention, there are provided the aforementioned method wherein tert-butanol is added in the ratio of 0.01 to 1,000 weight ppm based on the total weight of the cosmetic composition; the aforementioned method wherein the surfactant having an oxyethylene group in the molecule is one or more types of surfactants selected from the group consisting of the surfactants represented by the above general formulae (1), (2), and (3). According to further aspects, the present invention provides a suppressant of the smell change or the odor generation with passage of time in cosmetics comprising a surfactant having an oxyethylene group in the molecule, which comprises tert-butanol.

### BEST MODE FOR CARRYING OUT THE INVENTION

The cosmetic composition of the present invention is characterized in that said composition comprises (A) one or more types of surfactant selected from the group consisting of surfactants which have an oxyethylene group in the molecule; (B) tert-butanol, and the smell change or the odor generation with passage of time in said composition is suppressed by tert-butanol. The term “smell change or odor generation” should be construed in its broadest sense including alteration of smell in a composition; addition of bad smell; generation of some odor from an odorless composition, and should not be construed in any limitative sense. Suppression of the smell change or the odor generation includes reduction of the smell change or the odor generation, as well as substantially complete elimination of the smell change or the odor generation.

Type of the surfactant having an oxyethylene group is not limited as far as the surfactant has one or more oxyethylene groups in the molecule. The oxyethylene group contained in the molecule is preferred to be a polyoxyethylene group. More specifically, examples include a polyoxyethylenealkyl ether sulfate, a polyoxyethylenenonylphenyl ether sulfate, a polyoxyethylenealkylsulfo succinate, a polyoxyethylenealkyl ether acetate, a polyoxyethylenealkyl ether phosphoric acid, a polyoxyethylenealkylphenyl ether phosphoric acid, a fatty acid diethanolamide, a fatty acid monoethanolamide, a polyethylene glycol distearate, a polyoxyethylenealkyl ether, a polyoxyethylenenonylphenyl ether, a fatty acid polyoxyethylenealkyl ether, a fatty acid polyoxyethyleneglycerol ether, a polyoxyethylene hardened castor oil, a fatty acid polyoxyethylene hardened castor oil, a fatty acid polyoxyethylene sorbitan, or a polyoxyethylene-methylpolysiloxane copolymer. A single surfactant having an oxyethylene group may be used, or a combination of two or more surfactants may also be used.

Most preferred classes of the surfactant having an oxyethylene group are one or more types of surfactants selected from the group consisting of an alkyl ether sulfate represented by the following general formula (1):

[R¹(OCH₂CH₂)ₙ—OSO₃]—M+

(wherein R¹ represents a linear or branched alkyl group having 7 to 21 carbon atoms or a linear or branched alkenyl group having 7 to 21 carbon atoms, n represents an integer of 1 to 30, and M represents Na, K, NH₄, or triethanolamine);

an alkanolamide represented by the following general formula (2):

R²CO—NH(CH₂CH₂O)ₘH

(wherein R² represents a linear or branched alkyl group having 7 to 21 carbon atoms or a linear or branched alkenyl group having 7 to 21 carbon atoms, and m represents an integer of 1 to 10);

an N-acyl diethanolamide represented by the following general formula (3):

R³CO—N (CH₂CH₂OH)₂

(wherein R³ represents a linear or branched alkyl group having 7 to 21 carbon atoms or a linear or branched alkenyl group having 7 to 21 carbon atoms).

In the above general formulae, the alkyl group or the alkenyl group having 7 to 21 carbon atoms represented by R¹, R², and R³ may be linear or branched. When a branched alkyl group is used, the number of branch is not particularly limited. The number of double bonds in the alkenyl group is not particularly limited, and the number is generally about 1 to 4, and preferably about 1 to 2. When the alkenyl group has two or more double bonds, they may be conjugated double bonds.

In the general formula (1), R¹ is preferred to be a linear or branched alkyl group having 11 to 15 carbon atoms or a linear or branched alkenyl group having 11 to 15 carbon atoms, and M is preferred to be Na, NH₄, or triethanolamine. More specifically, for example, sodium polyoxyethylene (3) alkyl (12-14) ether sulfate, sodium polyoxyethylene (2) alkyl (12, 13) ether sulfate or the like is preferred.

In the general formula (2), R² is preferred to be a linear or branched alkyl group having 9 to 18 carbon atoms or a linear or branched alkenyl group having 9 to 18 carbon atoms. More preferably R² is a linear or branched alkyl group having 10 to 14 carbon atoms or a linear or branched alkenyl group having 10 to 14 carbon atoms and m is 1. More specifically, for example, castor oil fatty acid monoethanol amide or lauric acid monoethanol amide or the like is preferred.

In the general formula (3), R³ is preferred to be a linear or branched alkyl group having 9 to 18 carbon atoms or a linear or branched alkenyl group having 9 to 18 carbon atoms. More specifically, for example, castor oil fatty acid diethanol amide or lauric acid diethanol amide or the like is preferred.

A content of the surfactant having an oxyethylene group in the molecule in the cosmetic composition can be suitably determined by those ordinary skilled in the art depending on type of the cosmetic, desired properties and the like, and the content is not particularly limited. Generally, the content may be in the range of 0.1 to 50 weight %, preferably 1 to 20 weight % based on the total weight of the cosmetic composition. When the content of the aforementioned surfactant is less than 1 weight %, the problem of the smell change or the odor generation with passage of time is generally reduced, and thus the addition of tert-butanol may not be needed for solving the problem.

A content of tert-butanol in the cosmetic composition is not particularly limited. For example, 0.01 to 1,000 weight ppm based on the total weight of the composition is preferred. When the content of tert-butanol is less than 0.01 weight ppm, suppression of the smell change or the odor generation with passage of time may sometimes be insufficient. When the content is more than 1,000 weight ppm, odor inherent to tert-butanol may sometimes cause a problem.

Type of the cosmetic composition of the present invention is not particularly limited. For example, the composition may be used as hair washing agents such as a shampoo, a hair rinse, a 2-in-1 shampoo, and a conditioning shampoo, various hair cosmetics such as a hair lotion, a hair conditioner, a hair treatment, hair cream, a hair lacquer, a hair liquid, a hair wax, a hair water, a hair styling agent, a perm solution, a hair coloring, an acidic hair coloring, a hair manicure, or various skin cosmetics such as a skin lotion, a milky lotion, a facial washing agent, a makeup remover, a cleansing lotion, an emollient lotion, nourishing cream, emollient cream, massage cream, cleansing cream, a body shampoo, hand soap, solid soap, preshave cream, a tanning cosmetic, deodorant powder, a deodorant lotion, a deodorant spray, a makeup remover gel, a moisture gel, a moisturizing essence, an ultraviolet rays protective essence, shaving foam, face powder, a foundation, lip rouge, cheek rouge, eyeliner, eye shadow, an eyebrow pencil, and a bath preparation, or toothpaste.

For the preparation of the cosmetic composition of the present invention, one or more additives which are generally used for the preparation of cosmetic compositions may be used. The aforementioned additive can be used in a suitable amount in a range where the additive does not destroy the advantageous effect of the present invention. For example, additives such as surfactants such as an anionic surfactant, a cationic surfactant, an amphoteric surfactant, and a nonionic surfactant, waxes, a vegetable oil, animal oil and fat, a derivative of natural oil and fat, mineral oil and fat, a lower and higher fatty acid ester, a synthetic oil and fat such as N-acyl glutamic acid ester, a polymer material, an alcohol, a polyhydric alcohol, an extract, an amino acid, a nucleic acid, a vitamin, a hydrolyzed protein and their derivatives, a glycerylorate, an enzyme, an anti-inflammatory agent, a disinfectant, an antiseptic, an anti-oxidant, an absorbent of ultraviolet rays, a chelating agent, an antihidrotic, an oxidative dye, a pH modifier, a pearling agent, and a wetting agent can be used. A method for preparation of the cosmetic composition of the present invention is not particularly limited. The aforementioned ingredient (A) and ingredient (B) may be mixed by a suitable means, added and mixed optionally with one or more of the aforementioned additives, and thus, the cosmetic composition of the present invention can be easily prepared by the method well known to those skilled in the art. The order of addition of each ingredient and the procedure of the obtained composition are not particularly limited.

### EXAMPLES

The present invention will be explained more specifically by referring to examples. However, the scope of the present invention is not limited to these examples.

### Test Example 1

A shampoo of the formulation shown in Table 1 (shown in weight %, total amount 100%) was prepared by an ordinary method, and the odor after a 6-month storage at 40° C. was evaluated. The evaluation was conducted based on the following criteria to calculate average values. The average value of 2.5 or more was represented as inferior (×), 1.5 to 2.4 was represented as slightly inferior (Δ), 1.4 or less was represented as superior (◯). The results of the evaluation are shown in Table 1. Comparative Examples 2 and 3 were prepared as compositions which was free from a surfactant having polyoxyethylene group in the molecule.

[Criteria for Evaluation]

Odor After Storage

3: Significant smell change or odor generation compared to the smell before the storage

2: Little smell change or odor generation compared to the smell before the storage

1: No smell change or odor generation compared to the smell before the storage

TABLE 1

| Ingredient | Example 1 | Example 2 | Comparative Example 1 | Comparative Example 2 | Comparative Example 3 |
|---|---|---|---|---|---|
| Sodium polyoxyethylene (2) lauryl ether sulfate | 9.0 | 9.0 | 9.0 | | |
| Sodium lauryl sulfate | | | | 9.0 | 9.0 |
| Cocamidepropylbetaine | 4.5 | 4.5 | 4.5 | 4.5 | 4.5 |
| Sodium chloride | 1.0 | 1.0 | 1.0 | 1.0 | 1.0 |
| Sodium PCA | 1.0 | 1.0 | 1.0 | 1.0 | 1.0 |
| Butylene glycol | 3.0 | 3.0 | 3.0 | 3.0 | 3.0 |
| Polyguaternium-10 | 0.2 | 0.2 | 0.2 | 0.2 | 0.2 |
| Methylparaben | 0.2 | 0.2 | 0.2 | 0.2 | 0.2 |
| Tert-Butanol (0.1% aqueous solution) | 0.50 | 0.05 | | 0.50 | |
| Water | the rest | the rest | the rest | the rest | the rest |
| total | 100.0 | 100.0 | 100.0 | 100.0 | 100.0 |
| Odor evaluation after storage | ∘ | ∘ | x | ∘ | ∘ |

### Test Example 2

A body shampoo of the formulation shown in Table 2 (shown in weight %, total amount 100%) was prepared by an ordinary method, and the odor after a 6-month storage at 40° C. was evaluated. The evaluation was conducted based on the following criteria to calculate average values. The average value of 2.5 or more was represented as inferior (×), 1.5 to 2.4 was represented as slightly inferior (Δ), 1.4 or less was represented as superior (◯). The results of the evaluation are shown in Table 2.

[Criteria for Evaluation]

Odor After Storage

3: Significant smell change or odor generation compared to the smell before the storage

2: Little smell change or odor generation compared to the smell before the storage

1: No smell change or odor generation compared to the smell before the storage

TABLE 2

| Ingredient | Example 3 | Example 4 | Comparative Example 4 |
|---|---|---|---|
| Lauric acid | 6.0 | 6.0 | 6.0 |
| Myristic acid | 4.0 | 4.0 | 4.0 |
| Palmitic acid | 2.0 | 2.0 | 2.0 |
| Potassium hydroxide | 3.4 | 3.4 | 3.4 |
| Propylene glycol | 6.0 | 6.0 | 6.0 |
| Lauramide DEA | 3.0 | 3.0 | 3.0 |
| Cocamidepropylbetaine | 3.0 | 3.0 | 3.0 |
| Hydroxypropylmethylcellulose | 0.2 | 0.2 | 0.2 |
| Glycol distearate | 2.0 | 2.0 | 2.0 |
| Tert-Butanol (0.1% aqueous solution) | 0.05 | 0.005 | |
| Water | the rest | the rest | the rest |
| total | 100.0 | 100.0 | 100.0 |
| Odor evaluation after storage | ∘ | ∘ | x |

### Test Example 3

A milky lotion of the formulation shown in Table 3 (shown in weight %, total amount 100%) was prepared, and the odor after a 6-month storage at 40 ° C. was evaluated. The evaluation was conducted based on the following criteria to calculate average values. The average value of 2.5 or more was represented as inferior (×), 1.5 to 2.4 was represented as slightly inferior (Δ), 1.4 or less was represented as superior (◯). The results of the evaluation are shown in Table 3.

[Criteria for Evaluation]

Odor After Storage

3: Significant smell change or odor generation compared to the smell before the storage

2: Little smell change or odor generation compared to the smell before the storage

1: No smell change or odor generation compared to the smell before the storage

TABLE 3

| Ingredient | Example 5 | Comparative Example 5 |
|---|---|---|
| Liquid paraffin | 5.0 | 5.0 |
| Dioctyldodecyl lauroyl glutamate | 2.0 | 2.0 |
| Propylene glycol stearate | 0.5 | 0.5 |
| PEG-5 hydrogenated castor oil | 1.5 | 1.5 |
| PEG-5 glyceryl stearate | 2.5 | 2.5 |
| Carbomer | 0.2 | 0.2 |
| Sodium hydroxide | 0.08 | 0.08 |
| Butylene glycol | 5.0 | 5.0 |
| Methylparaben | 0.1 | 0.1 |
| Tert-Butanol (0.1% aqueous solution) | 0.02 | |
| Water | the rest | the rest |
| total | 100.0 | 100.0 |
| Odor evaluation after storage | ∘ | Δ |

### Test Example 4

A milky lotion of the formulation shown in Table 4 (shown in weight %, total amount 100%) was prepared, and the odor after a 6-month storage at 40° C. was evaluated. The evaluation was conducted based on the following criteria to calculate average values. The average value of 2.5 or more was represented as inferior (×), 1.5 to 2.4 was represented as slightly inferior (Δ), 1.4 or less was represented as superior (◯). The results of the evaluation are shown in Table 4.

[Criteria for Evaluating]

Odor After Storage

3: Significant smell change or odor generation compared to the smell before the storage

2: Little smell change or odor generation compared to the smell before the storage

1: No smell change or odor generation compared to the smell before the storage

TABLE 4

| Ingredient | Example 6 | Example 7 | Comparative Example 6 | Comparative Example 7 | Comparative Example 8 |
|---|---|---|---|---|---|
| Cyclomethicone | 20.0 | 20.0 | 20.0 | 20.0 | 20.0 |
| Polyoxyethylene-methylpolysiloxane co- | 3.0 | 3.0 | 3.0 | | |
| polymer | | | | | |
| Glyceryl diisostearate | | | | 3.0 | 3.0 |
| Micro Particle Titanium Dioxide | 10.0 | 10.0 | 10.0 | 10.0 | 10.0 |
| Sodium PCA | 1.0 | 1.0 | 1.0 | 1.0 | 1.0 |
| Butylene glycol | 10.0 | 10.0 | 10.0 | 10.0 | 10.0 |
| Phenoxyethanol | 0.2 | 0.2 | 0.2 | 0.2 | 0.2 |
| Tert-Butanol (0.1% aqueous solution) | 1.0 | 0.1 | | 0.1 | |
| Water | the rest | the rest | the rest | the rest | the rest |
| total | 100.0 | 100.0 | 100.0 | 100.0 | 100.0 |
| Odor evaluation after storage | ∘ | ∘ | x | ∘ | ∘ |

### Example 8

### Preparation of a Shower Gel

A shower gel was prepared according to the following formulation by an ordinary method. The obtained shower gel was free from smell change or odor generation with passage of time, and highly stable.

TABLE 5

| Ingredient | Example 8 |
|---|---|
| Ammonium polyoxyethylene (3) lauryl ether sulfate | 9.0 |
| Cocamidepropylbetaine | 4.5 |
| Disodium cocoyl glutamate | 0.5 |
| Laureth-2 | 1.5 |
| Sodium PCA | 1.0 |
| Polyguaternium-10 | 0.4 |
| Methylparaben | 0.2 |
| tert-Butanol (0.1% aqueous solution) | 1.0 |
| Fragrance | suitable amount |
| Water | the rest |
| total | 100.0 |

### Example 9

### Preparation of a Hair Conditioner

A hair conditioner was prepared according to the following formulation by an ordinary method. The obtained hair conditioner was free from smell change or odor generation with passage of time, and highly stable.

TABLE 6

| Ingredient | Example 9 |
|---|---|
| Steartrimonium chloride | 1.00 |
| Cetyl alcohol | 4.00 |
| Hexyldecyl isostearate | 2.00 |
| Dimethicone | 2.00 |
| Steareth-40 | 2.00 |
| Lactic acid | 0.01 |
| Sodium PCA | 1.00 |
| Disodium EDTA | 0.05 |
| Methylparaben | 0.20 |
| Tert-Butanol (0.1% aqueous solution) | 1.00 |
| Fragrance | suitable amount |
| Water | the rest |
| total | 100.00 |

### Example 10

### Preparation of Facial Cleansing Foam

Facial cleansing foam was prepared according to the following formulation by an ordinary method. The obtained facial cleansing foam was free from smell change or odor generation with passage of time, and highly stable.

TABLE 7

| Ingredient | Example 10 |
|---|---|
| Lauric acid | 3.00 |
| Myristic acid | 9.00 |
| Palmitic acid | 8.00 |
| Stearic acid | 10.00 |
| Glycerol | 25.00 |
| Butylene glycol | 5.00 |
| Cocamide MEA | 2.00 |
| Glyceryl stearate | 1.00 |
| Potassium hydroxide | 5.95 |
| Cocamidepropylbetaine | 1.00 |
| Potassium cocoyl glycinate | 3.00 |
| Methylparaben | 0.20 |
| Tert-Butanol (0.1% aqueous solution) | 1.00 |
| Fragrance | suitable amount |
| Water | the rest |
| total | 100.0 |

Industrial Applicability

In the cosmetic composition of the present invention, the smell change or odor generation with passage of time, which is caused by addition of a surfactant having oxyethylene group in the molecule, is suppressed. Therefore the composition has a feature of high storage stability.

What is claimed is:

1. A cosmetic composition which comprises the following ingredients:
(A) one or more types of surfactant selected from the group consisting of a surfactant which has an oxyethylene group in the molecule;
(B) tert-butanol.

2. The cosmetic composition according to claim 1, wherein the ratio of the surfactant having an oxyethylene group in the molecule is 1 to 20 weight % based on the total weight of the composition.

3. The cosmetic composition according to claim 1 or 2, wherein the surfactant having an oxyethylene group in the molecule is one or more types of surfactants selected from the group consisting of a surfactant represented by the following general formula (1):
[R¹(OCH₂CH₂)ₙ—OSO₃]⁻M+
wherein R¹ represents a linear or branched alkyl group having 7 to 21 carbon atoms or a linear or branched alkenyl group having 7 to 21 carbon atoms, n represents an integer of 1 to 30, and M represents Na, K, NH₄, or triethanolamine;
a surfactant represented by the following general formula (2):
R²CO—NH(CH₂CH₂O)ₘH
wherein R² represents a linear or branched alkyl group having 7 to 21 carbon atoms or a linear or branched alkenyl group having 7 to 21 carbon atoms, and m represents an integer of 1 to 10;
a surfactant represented by the following general formula (3):
R³CO—N (CH₂CH₂OH)₂
wherein R³ represents a linear or branched alkyl group having 7 to 21 carbon atoms or a linear or branched alkenyl group having 7 to 21 carbon atoms.

4. The cosmetic composition according to claim 1, wherein a ratio of tert-butanol is 0.01 to 1,000 ppm based on the total weight of the composition.

5. A method for suppressing smell change or odor generation with passage of time in a cosmetic comprising a surfactant having an oxyethylene group in the molecule, which comprises a step of adding tert-butanol to said cosmetic.

6. The method according to claim 5, wherein tert-butanol is added in a ratio of 0.01 to 1,000 ppm based on the total weight of the cosmetic composition.

7. A suppressant of smell change or odor generation with passage of time in a cosmetic comprising a surfactant having an oxyethylene group in the molecule, which comprises tert-butanol.
